(19) United States
(12) Patent Application Publication
(10) Pub. No.: US 2020/0166179 A1
(43) Pub. Date: May 28, 2020

(54) CONTAINER FOR STORING A COMPOSITION COMPRISING TETRAFLUOROPROPENE AND METHOD FOR STORING SAME

(71) Applicant: ARKEMA FRANCE, Colombes (FR)

(72) Inventors: Béatrice BOUSSAND, Sainte Foy Les Lyon (FR); Wissam RACHED, Chaponost (FR); Laurent WENDLINGER, Soucieu en Jarrest (FR)

(73) Assignee: Arkema France, Colombes (FR)

(21) Appl. No.: 16/086,184

(22) PCT Filed: Mar. 17, 2017
(86) PCT No.: PCT/FR2017/050618

§ 371 Date: Sep. 18, 2018

(30) Foreign Application Priority Data
Mar. 22, 2016 (FR) 1652437

(51) Int. Cl. F17C 1/00; C07C 21/18; F17C 13/08

(52) U.S. Cl. CPC F17C 1/00; C07C 21/18; F17C 13/08; F17C 2201/0104; F17C 2203/0607; F17C 2221/038; F17C 2203/0643; F17C 2270/05; F17C 2223/0146; F17C 2205/0107; F17C 2203/066

(57) ABSTRACT

The invention relates to a container housing a composition comprising tetrafluoropropene. The container is made from metal and includes an inner surface, said inner surface being in contact with the composition and being at least partially covered with a polyether- or polyol-type resin. Advantageously, the aforementioned composition comprises at least 15 wt.-% tetrafluoropropene relative to the total weight of the composition.

### TECHNICAL FIELD

The present invention relates to a method for storing a composition comprising tetrafluoropropene. The present invention also relates to a container for storing a composition comprising tetrafluoropropene. In particular, the present invention relates to a container and to a method for stably storing a composition comprising tetrafluoropropene.

### TECHNOLOGICAL BACKGROUND OF THE INVENTION

Over the past few years, 2,3,3,3-tetrafluoropropene (CF₃CF═CH₂, hereinafter also referred to as HFO-1234yf) has attracted attention as a new refrigerant for replacing refrigerants such as, for example, chlorofluorocarbons (CFCs), hydrochlorofluorocarbons (HCFCs) or hydrofluorocarbons (HFCs), which are greenhouse effect gases. In general, compounds of the hydrochloropropene or hydrofluoropropene type can be unstable and can generate the formation of coproducts in the presence of water, of acid or of oxygen. A method for stabilizing 1,1,2,3-tetrachloropropene in the presence of morpholine or a derivative thereof or of trialkyl phosphate is known in particular from U.S. Pat. No. 9,228,128.

HFO-1234yf is for its part generally stored in the gas state and/or in the liquid state and transported in a pressurized closed container. Under certain conditions, HFO-1234yf can degrade and/or polymerize in the presence of oxygen. US 2015/0051426 describes in particular the storage of HFO-1234yf in a container in which the oxygen concentration is maintained below 1000 ppm by volume in order to prevent degradation thereof and polymerization thereof.

The presence of solid deposits associated with the degradation of HFO-1234yf during storage thereof or transportation thereof can prevent the subsequent use thereof as a refrigerant. In addition, in the event of the presence of a stabilizer in the composition, it is necessary to remove the stabilizer before using the HFO-1234yf, which makes the use thereof more complex and increases the risks linked to the residual presence of the stabilizer.

There is therefore a need for the implementation of a storage method which makes it possible to avoid the drawbacks of the known methods.

### SUMMARY OF THE INVENTION

According to a first aspect, the invention provides a container containing a composition comprising tetrafluoropropene, said container being made from metal and comprising an inner surface, said inner surface in contact with said composition being at least partially covered with a polyether- or polyol-type resin; advantageously, said composition comprises at least 15% by weight of tetrafluoropropene relative to the total weight of the composition.

According to one preferred embodiment, the polyether- or polyol-type resin is derived from monomers comprising an oxirane or phenol functional group.

Preferably, the polyether- or polyol-type resin is derived from monomers comprising a siloxirane entity.

In particular, the polyether- or polyol-type resin is derived from monomers comprising a siloxirane entity of formula (I)

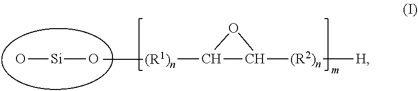

in which R¹ and R² are, independently of one another, for each unit n and m, a C₆-C₁₈ aryl, C₁-C₂₀ alkyl, C₃-C₂₀ cycloalkyl, C₃-C₂₀ cycloalkenyl, or C₂-C₂₀ alkenyl group, a carbonyl group of formula R³—C(O)—R⁴, an ester group of formula R³—C(O)—O—R⁴, an ether group of formula R³—O—R⁴; or an amine of formula R³—N—R⁴, R² which may also be an aldehyde group of formula R³—C(O)—H; R³ and R⁴ being chosen, independently of one another, from a C₆-C₁₈ aryl, C₁-C₂₀ alkyl, C₃-C₂₀ cycloalkyl, C₃-C₂₀ cycloalkenyl or C₂-C₂₀ alkenyl group;
m is an integer ranging from 1 to 30, advantageously from 1 to 20, preferably from 5 to 10; and
n is independently R¹ and R² an integer from 1 to 30, advantageously from 1 to 20, preferably from 5 to 10.

According to another preferred embodiment, the polyether- or polyol-type resin is derived from condensates of a compound A with a compound B, the compound A being an optionally substituted phenol compound and the compound B being a compound of formula R¹C(O)R² in which R¹ and R² are independently hydrogen, C₁-C₂₀ alkyl, C₆-C₂₀ aryl, C₃-C₂₀ cycloalkyl, or C₂-C₂₀ alkenyl.

Preferably, the compound A is phenol C₆H₅OH and the compound B is formaldehyde.

According to one preferred embodiment, at least 90% of said inner surface in contact with said composition is covered with said polyether- or polyol-type resin, advantageously at least 95% of said inner surface in contact with said composition is covered with said polyether- or polyol-type resin, preferably at least 98% of said inner surface in contact with said composition is covered with said polyether- or polyol-type resin, in particular at least 99% of said inner surface in contact with said composition is covered with said polyether- or polyol-type resin, more particularly the entire inner surface of the container in contact with said composition is covered with said polyether- or polyol-type resin.

According to one preferred embodiment, the container is made from steel.

According to one preferred embodiment, the container is made from carbon steel.

According to one preferred embodiment, the composition comprises less than 10 000 ppm by weight of water relative to the total weight of the composition, advantageously less than 5000 ppm by weight of water relative to the total weight of the composition, preferably less than 1000 ppm by weight of water relative to the total weight of the composition, in particular from 0.1 ppm to 100 ppm by weight of water relative to the total weight of the composition, more particularly from 1 to 20 ppm by weight of water relative to the total weight of the composition.

According to one preferred embodiment, the composition has an acid content, calculated in hydrochloric acid equivalent, of less than 100 ppm by weight relative to the total weight of the composition, advantageously less than 50 ppm by weight relative to the total weight of the composition, preferably less than 10 ppm by weight relative to the total weight of the composition, in particular from 0.01 ppm to 2 ppm by weight relative to the total weight of the composition.

According to one preferred embodiment, the composition is made up of a gas phase and a liquid phase.

According to one preferred embodiment, the composition comprises a gaseous air content of from 0.1% to 5% by volume relative to the total volume of the gas phase, advantageously from 0.5% to 3% by volume relative to the total volume of the gas phase, preferably from 0.01% to 2% by volume relative to the total volume of the gas phase.

According to one preferred embodiment, the composition comprises at least 90% by weight of tetrafluoropropene relative to the total weight of the composition, advantageously at least 95% by weight relative to the total weight of the composition, preferably at least 98% by weight relative to the total weight of the composition, in particular at least 99.5% by weight relative to the total weight of the composition.

According to one preferred embodiment, the tetrafluoropropene is 2,3,3,3-tetrafluoropropene or 1,3,3,3-tetrafluoropropene.

According to one preferred embodiment, the container is a closed container which withstands a test pressure, said test pressure being between 10 and 100 bar, advantageously between 15 and 70 bar, preferably between 20 and 60 bar, in particular from 40 to 50 bar.

According to one preferred embodiment, said container is cylindrical in shape and is mounted within a steel framework, said framework adhering to the dimensions of iso containers according to the standards ISO 1496-1:2013.

According to a second aspect, the invention provides a method for storing a composition comprising tetrafluoropropene, said method comprising the provision of a metal container, the inner surface of which is at least partially covered with a polyether- or polyol-type resin; and the filling of said container with a composition comprising tetrafluoropropene.

According to one preferred embodiment, the container is made from carbon steel and the coating is a polyether- or polyol-type resin, and the tetrafluoropropene is advantageously 2,3,3,3-tetrafluoropropene or 1,3,3,3-tetrafluoropropene.

According to a third aspect, the invention provides a device for loading an air-conditioning or refrigeration circuit, or for replacing a refrigerant mixture contained in an air-conditioning circuit or a refrigeration circuit, said device comprising a first container according to the invention and a second container comprising a lubricant; advantageously, said device also comprises one or more pipes capable of linking said first and second containers to the air-conditioning or refrigeration circuit; preferably, the air-conditioning circuit is an air-conditioning circuit of a vehicle.

### DETAILED DESCRIPTION OF THE INVENTION

According to a first aspect of the invention, a container is provided. The container may be of any shape; however, the container is preferably cylindrical. The container comprises an inner surface. Preferably, the container contains a composition comprising tetrafluoropropene. Said container is preferably made from metal. The container may optionally be compartmentalized, for example for containing, in each of the compartments, compositions comprising various amounts of tetrafluoropropene, or for containing for example, in one compartment, a composition comprising tetrafluoropropene and, in another compartment, another compound, for example a lubricant or another fluorinated compound such as a hydrofluorocarbon, a hydrochlorofluorocarbon or a hydrochlorocarbon.

The container can be suitable for transporting a composition comprising tetrafluoropropene and/or for storing a composition comprising tetrafluoropropene. The latter may for example be derived from a production unit for 2,3,3,3-tetrafluoropropene or for 1,3,3,3-tetrafluoropropene.

The container may be equipped with one or more valves for filling and/or emptying said container. For example, an inlet valve can be positioned so that the container is supplied with a stream containing a composition comprising tetrafluoropropene. The container may also be equipped with one or more outlet valves for emptying from said container the composition contained therein. For example, the composition may be emptied from the container in order to supply a device for purifying the composition comprising tetrafluoropropene or for supplying a second container according to the present invention and suitable for transporting a composition comprising tetrafluoropropene. The composition can also be emptied from the container in order to supply an air-conditioning circuit. Thus, the container according to the present invention may be a storage tank for storing a composition comprising tetrafluoropropene or a container which meets the ISO transportation standards. Said container is preferably a pressure-resistant closed container. The container may be cylinder or a cartridge or a small container which is pressure-resistant, the volume of which may preferably be less than 1 m³, advantageously less than 0.1 m³, preferably less than 0.01 m³. Alternatively, the container may be a storage or transportation tank, the volume of which may be greater than 0.5 m³, advantageously greater than 100 m³, preferably greater than 1000 m³, in particular greater than 5000 m³. The storage or transportation tank may preferably be spherical or cylindrical in shape. The container according to the present invention may comprise a thermally insulating wall.

Preferably, said inner surface of the container in contact with said composition comprising tetrafluoropropene is at least partially covered with a polyether- or polyol-type resin.

According to one preferred embodiment, at least 90% of said inner surface in contact with said composition may be covered with a polyether- or polyol-type resin, advantageously at least 95% of said inner surface in contact with said composition may be covered with a polyether- or polyol-type resin, preferably at least 98% of said inner surface in contact with said composition may be covered with a polyether- or polyol-type resin, in particular at least 99% of said inner surface in contact with said composition may be covered with a polyether- or polyol-type resin. More particularly, the entire inner surface in contact with said composition contained in said container may be covered with a polyether- or polyol-type resin. This makes it possible to minimize the contacts between said composition and the steel. This is because prolonged contact between the composition comprising tetrafluoropropene and the steel, in particular the iron contained in the steel, can promote the formation of hydrogen, in particular if the composition comprises water or hydrochloric acid.

According to one preferred embodiment, the polyether- or polyol-type resin is derived from monomers comprising an oxirane or phenol functional group.

Advantageously, the oxirane functional group can be bonded to one or more metal oxides. The metal oxide may be silica, aluminum oxide, magnesium oxide, titanium oxide, antimony oxide, zinc oxide, iron oxide or molybdenum oxide. Preferably, the oxirane functional group can be bonded to aluminum oxide or silica.

According to one preferred embodiment, the polyether- or polyol-type resin is derived from monomers comprising a siloxirane entity.

Preferably, the polyether- or polyol-type resin is derived from monomers comprising a siloxirane entity of formula (I)

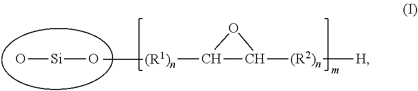

in which R¹ and R² are, independently of one another, for each unit n and m, a C₆-C₁₈ aryl, C₁-C₂₀ alkyl, C₃-C₂₀ cycloalkyl, C₃-C₂₀ cycloalkenyl, or C₂-C₂₀ alkenyl group, a carbonyl group of formula R³—C(O)—R⁴, an ester group of formula R³—C(O)—O—R⁴, an ether group of formula R³—O—R⁴; or an amine of formula R³—N—R⁴, R² which may also be an aldehyde group of formula R³—C(O)—H; R³ and R⁴ being chosen, independently of one another, from a C₆-C₁₈ aryl, C₁-C₂₀ alkyl, C₃-C₂₀ cycloalkyl, C₃-C₂₀ cycloalkenyl or C₂-C₂₀ alkenyl group;
m is an integer ranging from 1 to 30, advantageously from 1 to 20, preferably from 5 to 10; and
n is independently R¹ and R² an integer from 1 to 30, advantageously from 1 to 20, preferably from 5 to 10.

The term “alkyl” denotes a monovalent radical derived from a linear or branched alkane, comprising from 1 to 20 carbon atoms. The term “cycloalkyl” denotes a monovalent radical derived from a cycloalkane, comprising from 3 to 20 carbon atoms. The term “aryl” denotes a monovalent radical derived from an arene, comprising from 6 to 18 carbon atoms. The term “alkenyl” denotes a monovalent radical of 2 to 20 carbon atoms and at least one carbon-carbon double bond. The term “halogen” refers to an —F, —Cl, —Br or —I group. The term “cycloalkenyl” refers to a monovalent radical derived from a cycloalkene comprising from 3 to 20 carbon atoms. The C₁-C₂₀ alkyl, C₂-C₂₀ alkenyl, C₂-C₂₀ alkynyl, C₃-C₂₀ cycloalkyl, C₃-C₂₀ cycloalkenyl and C₆-C₁₈ aryl groups may be optionally substituted with one or more —OH, halogen, —NR^{a}C(O)R^{b}, —C(O)NR^{a}R^{b}, —NR^{a}R^{b}, —OR^{a}, —CO₂R^{a}, —OC(O)OR^{a}, —OC(O)R^{a}, —C(O)H or —C(O)R^{a} substituents in which R^{a} and R^{b} are, independently of one other, hydrogen, unsubstituted C₁-C₂₀ alkyl, unsubstituted C₂-C₂₀ alkenyl, unsubstituted C₃-C₂₀ cycloalkyl, unsubstituted C₃-C₂₀ cycloalkenyl, or unsubstituted C₆-C₁₈ aryl. In the substituents —NR^{a}R^{b}, R^{a} and R^{b} may form, with the nitrogen atom to which they are attached, a saturated or unsaturated, aromatic or non-aromatic, 5- to 10-membered heterocycle.

According to another preferred embodiment, the polyether- or polyol-type resin is derived from condensates of a compound A with a compound B, the compound A being an optionally substituted phenol compound and the compound B being a compound of formula R⁵C(O)R⁶ in which R⁵ and R⁶ are independently hydrogen, C₁-C₂₀ alkyl, C₆-C₂₀ aryl, C₃-C₂₀ cycloalkyl, or C₂-C₂₀ alkenyl. The substituted phenol compound can be substituted with any one of the substituents mentioned above. Preferably, the compound A is an unsubstituted phenol. Advantageously, the compound B is a compound of formula R⁵C(O)R⁶ in which R⁵ and R⁶ are independently hydrogen, C₁-C₁₀ alkyl, C₆-C₁₀ aryl, C₃-C₁₀ cycloalkyl, or C₂-C₁₀ alkenyl. Preferably, the compound B is a compound of formula R⁵C(O)R⁶ in which R⁵ and R⁶ are independently hydrogen, C₁-C₅ alkyl, C₆-C₁₀ aryl, C₃-C₆ cycloalkyl, or C₂-C₅ alkenyl. In particular, the compound B is a compound of formula R⁵C(O)R⁶ in which R⁵ and R⁶ are hydrogen.

Preferably, the compound A is phenol C₆H₅OH and the compound B is formaldehyde.

According to one preferred embodiment, the container may be made from steel. Advantageously, the container is made from carbon steel or from stainless steel. Preferably, the container is made from carbon steel.

Advantageously, said composition comprises at least 15% by weight of tetrafluoropropene relative to the total weight of the composition. Preferably, said composition comprises at least 40% by weight of tetrafluoropropene relative to the total weight of the composition, more preferentially at least 60% by weight of tetrafluoropropene relative to the total weight of the composition, in particular at least 70% by weight of tetrafluoropropene relative to the total weight of the composition, more particularly at least 80% by weight of tetrafluoropropene relative to the total weight of the composition.

According to one particular embodiment, said composition may comprise at least 90% by weight of tetrafluoropropene relative to the total weight of the composition, advantageously at least 95% by weight relative to the total weight of the composition, preferably at least 98% by weight relative to the total weight of the composition, in particular at least 99.5% by weight relative to the total weight of the composition.

Preferably, the tetrafluoropropene may be 2,3,3,3-tetrafluoropropene and/or 1,3,3,3-tetrafluoropropene.

Thus, said composition may comprise at least 15% by weight of 2,3,3,3-tetrafluoropropene relative to the total weight of the composition. Preferably, said composition comprises at least 40% by weight of 2,3,3,3-tetrafluoropropene relative to the total weight of the composition, more preferentially at least 60% by weight of 2,3,3,3-tetrafluoropropene relative to the total weight of the composition, in particular at least 70% by weight of 2,3,3,3-tetrafluoropropene relative to the total weight of the composition, more particularly at least 80% by weight of 2,3,3,3-tetrafluoropropene relative to the total weight of the composition. According to one particular embodiment, said composition may comprise at least 90% by weight of 2,3,3,3-tetrafluoropropene relative to the total weight of the composition, advantageously at least 95% by weight of 2,3,3,3-tetrafluoropropene relative to the total weight of the composition, preferably at least 98% by weight of 2,3,3,3-tetrafluoropropene relative to the total weight of the composition, in particular at least 99.5% by weight of 2,3,3,3-tetrafluoropropene relative to the total weight of the composition.

Alternatively, said composition may comprise at least 15% by weight of 1,3,3,3-tetrafluoropropene relative to the total weight of the composition. Preferably, said composition comprises at least 40% by weight of 1,3,3,3-tetrafluoropropene relative to the total weight of the composition, more preferentially at least 60% by weight of 1,3,3,3-tetrafluoropropene relative to the total weight of the composition, in particular at least 70% by weight of 1,3,3,3-tetrafluoropropene relative to the total weight of the composition, more particularly at least 80% by weight of 1,3,3,3-tetrafluoropropene relative to the total weight of the composition. According to one particular embodiment, said composition may comprise at least 90% by weight of 1,3,3,3-tetrafluoropropene relative to the total weight of the composition, advantageously at least 95% by weight of 1,3,3,3-tetrafluoropropene relative to the total weight of the composition, preferably at least 98% by weight of 1,3,3,3-tetrafluoropropene relative to the total weight of the composition, in particular at least 99.5% by weight of 1,3,3,3-tetrafluoropropene relative to the total weight of the composition.

Said composition contained in the container may comprise less than 10 000 ppm by weight of water relative to the total weight of the composition, advantageously less than 5000 ppm by weight of water relative to the total weight of the composition, preferably less than 1000 ppm by weight of water relative to the total weight of the composition, more preferentially less than 500 ppm by weight of water relative to the total weight of the composition, in particular less than 100 ppm by weight of water relative to the total weight of the composition, more particularly less than 50 ppm by weight relative to the total weight of the composition.

Advantageously, said composition may comprise from 0.01 to 500 ppm by weight of water relative to the total weight of the composition, preferably from 0.05 to 250 ppm by weight of water relative to the total weight of the composition, in particular from 0.1 to 100 ppm by weight of water relative to the total weight of the composition, more particularly from 1 to 20 ppm of water relative to the total weight of the composition.

According to one preferred embodiment, said composition may comprise a gaseous air content of from 0.1% to 5% by volume relative to the total volume of the gas phase, advantageously from 0.5% to 3%, preferably from 0.01% to 2% by volume relative to the total volume of the gas phase.

Preferably, said composition may comprise less than 10 000 ppm by weight of water relative to the total weight of the composition, advantageously less than 5000 ppm by weight of water relative to the total weight of the composition, preferably less than 1000 ppm by weight of water relative to the total weight of the composition, more preferentially less than 500 ppm by weight of water relative to the total weight of the composition, in particular less than 100 ppm by weight of water relative to the total weight of the composition, more particularly less than 50 ppm by weight of water relative to the total weight of the composition; and a gaseous air content of from 0.1% to 5% by volume relative to the total volume of the gas phase, advantageously from 0.5% to 3% by volume relative to the total volume of the gas phase, preferably from 0.01% to 2% by volume relative to the total volume of the gas phase.

In particular, said composition may comprise from 0.01 to 500 ppm by weight of water relative to the total weight of the composition, preferably from 0.05 to 250 ppm by weight of water relative to the total weight of the composition, in particular from 0.1 ppm to 100 ppm by weight of water relative to the total weight of the composition, more particularly from 1 to 20 ppm by weight of water relative to the total weight of the composition; and a gaseous air content of from 0.1% to 5% by volume relative to the total volume of the gas phase, advantageously from 0.5% to 3% by volume relative to the total volume of the gas phase, preferably from 0.01% to 2% by volume relative to the total volume of the gas phase.

According to one preferred embodiment, the composition may have an acid content, calculated in hydrochloric acid equivalent, of less than 100 ppm by weight of acid relative to the total weight of the composition, advantageously less than 50 ppm by weight of acid relative to the total weight of the composition, preferably less than 10 ppm by weight of acid relative to the total weight of the composition, in particular from 0.01 ppm to 2 ppm by weight of acid relative to the total weight of the composition.

Advantageously, said composition may have an acid content, calculated in hydrochloric acid equivalent, of less than 100 ppm by weight relative to the total weight of the composition, advantageously less than 50 ppm by weight relative to the total weight of the composition, preferably less than 10 ppm by weight relative to the total weight of the composition, in particular from 0.01 ppm to 2 ppm by weight relative to the total weight of the composition; and may comprise less than 10 000 ppm by weight of water relative to the total weight of the composition, advantageously less than 5000 ppm by weight relative to the total weight of the composition, preferably less than 1000 ppm by weight of water relative to the total weight of the composition, more preferentially less than 500 ppm by weight of water relative to the total weight of the composition, in particular less than 100 ppm by weight of water relative to the total weight of the composition, more particularly less than 50 ppm by weight of water relative to the total weight of the composition. Alternatively, said composition may have an acid content, calculated in hydrochloric acid equivalent, of less than 100 ppm by weight relative to the total weight of the composition, advantageously less than 50 ppm by weight relative to the total weight of the composition, preferably less than 10 ppm by weight relative to the total weight of the composition, in particular from 0.01 ppm to 2 ppm by weight relative to the total weight of the composition; and may comprise from 0.01 to 500 ppm of water by weight relative to the total weight of the composition, preferably from 0.05 to 250 ppm by weight relative to the total weight of the composition, in particular from 0.1 ppm to 100 ppm by weight relative to the total weight of the composition, more particularly from 1 to 20 ppm by weight relative to the total weight of the composition.

Said composition may have an acid content, calculated in hydrochloric acid equivalent, of less than 100 ppm by weight relative to the total weight of the composition, advantageously less than 50 ppm by weight relative to the total weight of the composition, preferably less than 10 ppm by weight relative to the total weight of the composition, in particular from 0.01 ppm to 2 ppm by weight relative to the total weight of the composition; and a gaseous air content of from 0.1% to 5% by volume relative to the total volume of the gas phase, advantageously from 0.5% to 3% by volume relative to the total volume of the gas phase, preferably from 0.01% to 2% by volume relative to the total volume of the gas phase.

Preferably, said composition may have an acid content, calculated in hydrochloric acid equivalent, of less than 100 ppm by weight relative to the total weight of the composition, advantageously less than 50 ppm by weight relative to the total weight of the composition, preferably less than 10 ppm by weight relative to the total weight of the composition, in particular from 0.01 ppm to 2 ppm by weight relative to the total weight of the composition; and a gaseous air content of from 0.1% to 5% by volume relative to the total volume of the gas phase, advantageously from 0.5% to 3% by volume relative to the total volume of the gas phase, preferably from 0.01% 2% by volume relative to the total volume of the gas phase; and may comprise from 0.01 to 500 ppm by weight of water relative to the total weight of the composition, preferably from 0.05 to 250 ppm by weight of water relative to the total weight of the composition, in particular from 0.1 ppm to 100 ppm by weight of water relative to the total weight of the composition, more particularly from 1 to 20 ppm by weight of water relative to the total weight of the composition.

Advantageously, said composition may have an acid content, calculated in hydrochloric acid equivalent, of less than 100 ppm by weight relative to the total weight of the composition, advantageously less than 50 ppm by weight relative to the total weight of the composition, preferably less than 10 ppm by weight relative to the total weight of the composition, in particular from 0.01 ppm to 2 ppm by weight relative to the total weight of the composition; and a gaseous air content of from 0.1% to 5% by volume relative to the total volume of the gas phase, advantageously from 0.5% to 3% by volume relative to the total volume of the gas phase, preferably from 0.01% to 2% by volume relative to the total volume of the gas phase; and may comprise less than 10 000 ppm by weight of water relative to the total weight of the composition, advantageously less than 5000 ppm by weight of water relative to the total weight of the composition, preferably less than 1000 ppm by weight of water relative to the total weight of the composition, more preferentially less than 500 ppm by weight of water relative to the total weight of the composition, in particular less than 100 ppm by weight of water relative to the total weight of the composition, more particularly less than 50 ppm by weight of water relative to the total weight of the composition.

Said container may be a closed container which withstands a test pressure, said test pressure may be between 10 and 100 bar, advantageously between 15 and 70 bar, preferably between 20 and 60 bar, in particular between 40 and 50 bar.

Preferably, when said composition is contained in the container, said composition may be made up of a gas phase and a liquid phase. In particular, the gas phase and the liquid phase can have the same chemical composition, that is to say that the proportion of tetrafluoropropene in the liquid phase and in the gas phase is substantially identical, when said composition comprises at least 95% by weight of tetrafluoropropene, preferably at least 98% by weight of tetrafluoropropene, in particular at least 99.5% by weight of tetrafluoropropene.

According to one preferred embodiment, said container is cylindrical in shape and is mounted within a steel framework, said framework adhering to the dimensions of iso containers according to the standards ISO 1496-1:2013.

According to another preferred embodiment, said container is a cartridge which withstands said test pressure mentioned above. Preferably, the internal volume of the cartridge containing a composition comprising 2,3,3,3-tetrafluoropropene or 1,3,3,3-tetrafluoropropene is less than 1 m³, advantageously less than 0.1 m³, preferably less than 0.01 m³.

According to a second aspect, the invention provides a method for storing a composition comprising tetrafluoropropene, said method comprising the provision of a metal container, the inner surface of which is at least partially covered with a polyether- or polyol-type resin; and the filling of said container with a composition comprising tetrafluoropropene. The container and the composition are as defined in the first aspect of the present invention.

According to one preferred embodiment, the container is made from carbon steel and the resin is as defined above in the detailed description of the invention, and advantageously the tetrafluoropropene is 2,3,3,3-tetrafluoropropene and/or 1,3,3,3-tetrafluoropropene.

According to another aspect of the present invention, a device is provided. Said device comprises a container, preferably a cylinder or cartridge or a small container, according to the present invention, and a second container comprising a lubricant. The device according to the invention can thus be suitable for preparing a refrigerant mixture comprising a lubricant and tetrafluoropropene. The device according to the invention can also be suitable for loading an air-conditioning circuit, preferably an automobile air-conditioning circuit, or a refrigeration circuit. Preferably, the lubricant can be suitable for automobile air conditioning. By way of lubricants, use may in particular be made of polyalkylene glycols, polyol esters and/or polyvinyl ethers. Said device also comprises connectors linking, optionally independently, each of the containers to an air-conditioning or refrigeration container or circuit in which the lubricant of said second container is mixed with said composition comprising tetrafluoropropene contained in said container according to the present invention.

### Examples

Carbon steel coupons totally covered with a resin derived from a monomer comprising a siloxirane-type functional group or with a resin derived from condensates of phenol and of formaldehyde are introduced into sealed glass tubes. 5000 ppm of 0.1 N hydrochloric acid are introduced into each tube by weighing. The sealed tube is placed in dry ice in order to facilitate the pulling and the transfer of the refrigerant fluid in said tube. The cylinder containing the refrigerant fluid is connected to the sealed tube, a vacuum is pulled in the entire device and the leaktightness is verified. The fluid is then expanded in a buffer cylinder of known volume. A determined weight of the fluid can thus be introduced into the sealed tube. This weight is calculated as a function of the pressure difference, the temperature and the buffer volume. It is verified using a manometer that the product has indeed been transferred, and the tube is sealed. The tube thus sealed is weighed then placed in a metal sheath, and placed in an incubator at 85° C. for 14 days or 1 month. In this example, the refrigerant fluid is 2,3,3,3-tetrafluoropropene.

Visual observation of the tubes does not show any change in color of the liquid 2,3,3,3-tetrafluoropropene, whether after 14 days or 1 month. No coproduct is detected in the refrigerant fluid. The resin derived from a monomer comprising a siloxirane-type functional group shows a slight coloration after one month. The resin derived from condensates of phenol with formaldehyde, for its part, shows no change in appearance after 1 month. A container coated with a resin derived from condensates of phenol with formaldehyde allows prolonged storage or transportation of tetrafluoropropene without degradation.

1-20. (canceled)

21. A container containing a composition comprising tetrafluoropropene, said container being made from metal and comprising an inner surface, said inner surface in contact with said composition being at least partially covered with a polyether- or polyol-type resin; wherein said composition comprises at least 15% by weight of tetrafluoropropene relative to the total weight of the composition.

22. The container as claimed in claim 21, wherein the polyether- or polyol-type resin is derived from monomers comprising an oxirane or phenol functional group.

23. The container as claimed in claim 21, wherein the polyether- or polyol-type resin is derived from monomers comprising a siloxirane entity.

24. The container as claimed in claim 23, wherein the polyether- or polyol-type resin is derived from monomers comprising a siloxirane entity of formula (I) 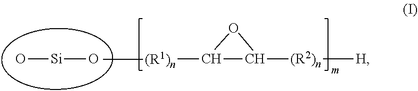
in which R¹ and R² are, independently of one another, for each unit n and m, a C₆-C₁₈ aryl, C₁-C₂₀ alkyl, C₃-C₂₀ cycloalkyl, C₃-C₂₀ cycloalkenyl, or C₂-C₂₀ alkenyl group, a carbonyl group of formula R³—C(O)—R⁴, an ester group of formula R³—C(O)—O—R⁴, an ether group of formula R³—O—R⁴; or an amine of formula R³—N—R⁴, R² which may also be an aldehyde group of formula R³—C(O)—H; R³ and R⁴ being chosen, independently of one another, from a C₆-C₁₈ aryl, C₁-C₂₀ alkyl, C₃-C₂₀ cycloalkyl, C₃-C₂₀ cycloalkenyl or C₂-C₂₀ alkenyl group;
m is an integer ranging from 1 to 30; and
n is independently R¹ and R² an integer from 1 to 30.

25. The container as claimed in claim 21, wherein the polyether- or polyol-type resin is derived from condensates of a compound A with a compound B, the compound A being an optionally substituted phenol compound and the compound B being a compound of formula R⁵C(O)R⁶ in which R⁵ and R⁶ are independently hydrogen, C₁-C₂₀ alkyl, C₆-C₂₀ aryl, C₃-C₂₀ cycloalkyl, or C₂-C₂₀ alkenyl.

26. The container as claimed in claim 25, wherein the compound A is phenol C₆H₅OH and the compound B is formaldehyde.

27. The container as claimed in claim 21, wherein at least 90% of said inner surface in contact with said composition is covered with said polyether- or polyol-type resin.

28. The container as claimed in claim 21, wherein the container is made from steel.

29. The container as claimed in claim 21, wherein the container is made from carbon steel.

30. The container as claimed in claim 21, wherein the composition comprises less than 10 000 ppm by weight of water relative to the total weight of the composition.

31. The container as claimed in claim 21, wherein the composition is made up of a gas phase and a liquid phase.

32. The container as claimed in claim 31, wherein the composition comprises a gaseous air content of from 0.1% to 5% by volume relative to the total volume of the gas phase.

33. The container as claimed in claim 21, wherein the composition has an acid content, calculated in hydrochloric acid equivalent, of less than 100 ppm by weight relative to the total weight of the composition.

34. The container as claimed in claim 21, wherein the composition comprises at least 90% by weight of tetrafluoropropene relative to the total weight of the composition.

35. The container as claimed in claim 21, wherein the tetrafluoropropene comprises 2,3,3,3-tetrafluoropropene or 1,3,3,3-tetrafluoropropene.

36. The container as claimed in claim 21, wherein the container is a closed container which withstands a test pressure, said test pressure being between 10 and 100 bar.

37. The container as claimed in claim 21, wherein said container is cylindrical in shape and is mounted within a steel framework, said framework adhering to the dimensions of iso containers according to the standards ISO 1496-1:2013.

38. A method for storing a composition comprising tetrafluoropropene, said method comprising providing a metal container, the inner surface of which is at least partially covered with a polyether- or polyol-type resin; and filling said container with a composition comprising tetrafluoropropene.

39. The method as claimed in claim 38, wherein the container is made from carbon steel, the coating is a polyether- or polyol-type resin derived from monomers comprising an oxirane or phenol functional group, and the tetrafluoropropene comprises 2,3,3,3-tetrafluoropropene or 1,3,3,3-tetrafluoropropene.

40. A device for loading an air-conditioning circuit or for replacing a refrigerant mixture contained in an air-conditioning or refrigeration circuit, said device comprising a first container as claimed in claim 21, a second container comprising a lubricant, and one or more pipes capable of linking said first and second containers to the air-conditioning-conditioning or refrigeration circuit.
